(19) United States
(12) Patent Application Publication
(10) Pub. No.: US 2016/0008493 A1
(43) Pub. Date: Jan. 14, 2016

(54) RADIOACTIVE SUBSTRATES FOR ALDEHYDE DEHYDROGENASE

(71) Applicant: THE JOHNS HOPKINS UNIVERSITY, Baltimore, MD (US)

(72) Inventors: MARTIN C. POMPER, BALTIMORE, MD (US); HAOFAN WANG, ROCKVILLE, MD (US); IL MINN, ELLICOTT CITY, MD (US); RONNIE C. MEASE, FAIRFAX, VA (US)

(73) Assignee: THE JOHNS HOPKINS UNIVERSITY, BALTIMORE, MD (US)

(21) Appl. No.: 14/777,117

(22) PCT Filed: Mar. 17, 2014
(86) PCT No.: PCT/US2014/030274

§ 371 Date: Sep. 15, 2015

Related U.S. Application Data
(60) Provisional application No. 61/791,272, filed on Mar. 15, 2013.

(51) Int. Cl. A61K 51/04; C07C 235/42; G01N 33/60

(52) U.S. Cl. CPC A61K 51/04; G01N 33/60; C07C 235/42; G01N 2333/90203

(57) ABSTRACT

A detectable substrate for aldehyde dehydrogenase (ALDH) can include a radiolabel. When acted upon by ALDH in an ALDH-expressing cell, e.g., cancer cells, the radiolabeled substrate accumulates in the ALDH-expressing cell. ALDH-expressing cells can be distinguished by the accumulated radioactivity. When combined with suitable imaging technique, the detectable substrate can be used for in vivo imaging of cancer cells.

### CROSS-REFERENCE TO RELATED APPLICATION

This application claims the benefit of U.S. Provisional Application No. 61/791,272, filed Mar. 15, 2013; which is incorporated herein by reference in its entirety.

### BACKGROUND

Aldehyde dehydrogenase (ALDH) is an evolutionarily conserved enzyme with pyridine nucleotide dependent oxidoreductase activity that performs a variety of critical cellular processes. These include production of retinoic acid essential for mammalian development, metabolism of fats and amino acids, and detoxification of endogenous and exogenous sources of hazardous aldehyde byproducts. Twenty human ALDH genes have been identified and many of their functions are still unknown. For the past two decades ALDH has been studied as a potential universal marker for normal and cancer stem cells as certain isoenzymes of the ALDH superfamily have been identified as key elements of these cells. For example, Aldh1a1 and Aldh3a1 have been implicated in the protection of stem cells from cytotoxic drugs. ALDHP^{pos} stem cells have been used as resources for regenerative medicine in preclinical models and in an ongoing clinical trial for ischemic cardiomyopathy (clinicatrial.gov, NCT00314366). ALDH1 has been identified as a marker used to isolate cancer stem cells of various human malignancies including bladder, breast, cervical, colon, head and neck, liver, lung, pancreas, prostate, and ovary.

Since these normal and cancer stem cells are very rare, methods to identify and isolate viable, functionally active ALDHP^{pos} cells are needed to characterize them. Detectable ALDH substrates reveal those cells in a population that have ALDH activity. Furthermore, because ALDH is a marker of cancer stem cells, compounds that become localized in ALDHP^{pos} cells can be used for imaging cancer stem cells and associated tumors in vivo. Compounds that become localized in ALDHP^{pos} cells can be used to deliver therapeutic radiation to cancer stem cells (and associated cancers).

### SUMMARY

Selection of cells positive for aldehyde dehydrogenase (ALDH) is difficult with existing reagents. Radioactive detectable substrates for ALDH are useful in labeling viable ALDHP^{pos} cells. The substrates also can become localized in ALDHP^{pos} cancer stem cells, such that the radioactivity is delivered to and localized in the cancer stem cells. A simple radiosynthesis is provided for practical preparation and use of the radioactive substrates.

In one aspect, a detectable substrate for ALDH includes a compound of formula (I):

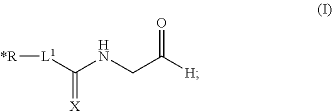

where X can be O or S: L¹ can be a linker moiety; *R can be a radiolabeled moiety; and provided that the compound is not [¹²⁵I]N-(formylmethyl)-5-iodopyridine-3-carboxamide or [¹²⁵I]4-(diethylamino)-3-iodobenzaldehyde.

In some aspects, L¹ can be a bond; a C₁-C₁₀ alkylene moiety optionally substituted with from one to five substituents individually selected from H, halogen, alkyl, cycloalkyl, —OH, —O-alkyl, nitro, cyano, aryl, or heteroaryl, and optionally interrupted by one to three groups individually selected from —O—, —S—, —C(O)—, C(S)—, —N(R^{a})—, and —C(O)N(R^{a})—; or a C₁-C₁₀ alkenylene moiety optionally substituted with from one to five substituents individually selected from H, halogen, alkyl, cycloalkyl, —OH, —O-alkyl, nitro, cyano, aryl, or heteroaryl, and optionally interrupted by one to three groups individually selected from —O—, —S—, —C(O)—, —C(S)—, —N(R^{a})—, and —C(O)N(R^{a})—; wherein each R^{a}, individually, can be H, alkyl, or aryl.

*R can have the formula:

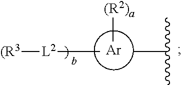

where each R², individually, can be H, halogen, —OH, nitro, cyano, alkyl, —O-alkyl, alkenyl, alkynyl, cycloalkyl, heterocyclyl, aryl, or heteroaryl, wherein each of alkyl, alkenyl, alkynyl, cycloalkyl, heterocyclyl, aryl and heteroaryl is optionally substituted by one to five substitutents independently selected from H, halogen, —OH, nitro, cyano, alkyl, —O-alkyl, alkenyl, or R³; a can be 0, 1, 2, 3, 4, or 5; L² can be: (a) a bond; (b) a C₁-C₁₀ alkylene moiety optionally substituted with from one to five substituents individually selected from H, halogen, alkyl, cycloalkyl, —OH, —O-alkyl, nitro, cyano, aryl, or heteroaryl and optionally interrupted by one to three group individually selected from —O—, —S—, —C(O)—, —C(S)—, —N(R^{a})—, and —C(O)N(R^{a})—; (c) a C₁-C₁₀ alkenylene moiety optionally substituted with from one to five substituents individually selected from H, halogen, alkyl, cycloalkyl, —OH, —O-alkyl, nitro, cyano, aryl, or heteroaryl, and optionally interrupted by one to three groups individually selected from —O—, —SO—, —C(O)—, —C(S)—, —N(R^{a})—, and —C(O)N(R^{a})—; or (d) a C₆₋₁₀ arylene moiety optionally substituted with from one to five substituents individually selected from H, halogen, alkyl, cycloalkyl, —OH, —O-alkyl, nitro, cyano, aryl, or heteroaryl; where each R^{a}, individually, can be H, alkyl, or aryl; each R³, independently, can be a radioisotope; and b can be 1, 2, 3, 4, or 5.

In some embodiments, Ar can be monocyclic aryl or heteroaryl; a can be 0 or 1; b can be 1; and L² can be a bond. Each R³, independently, can be ¹⁸F, ¹²³I, ¹²⁴I, ¹²⁵I, ¹²⁶I, ¹³¹I, ⁷⁵Br, ⁷⁶Br, ⁷⁷Br, ⁸⁰Br, ^{80m}Br, ⁸²Br, ⁸³Br or ²¹¹At. L¹ can be a bond or a C₁-C₁₀ alkylene moiety optionally substituted with from one to five substituents individually selected from H, halogen, alkyl, cycloalkyl, —OH, —O-alkyl, nitro, cyano, aryl, or heteroaryl, and optionally interrupted by one to three groups individually selected from —O—, —S—, —C(O)—, —C(S)—, —N(R^{a})—, and —C(O)N(R^{a})—.

In another aspect, a detectable substrate for ALDH includes a compound having the formula:

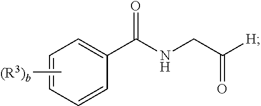

where each R³, independently, is a radioisotope, and b is 1, 2, 3, 4, or 5.

In some aspects, R³ can be ¹²⁵I and b can be 1 and the detectable substrate can have the formula:

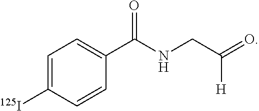 [SMILES: O=CCNC(=O)c1ccc([125I])cc1]

In another aspect, a method of distinguishing ALDH-expressing cells in a population of cells includes exposing the population of cells to a detectable substrate for ALDH as described hereinabove; measuring radioactivity from the cells; and identifying cells exhibiting increased radioactivity from the detectable substrate.

In some aspects, the method can further include, prior to measuring radioactivity from the cells: converting the detectable substrate to the corresponding carboxylic acid within cells expressing ALDH; and retaining and accumulating the corresponding carboxylic acid within cells expressing ALDH. Measuring radioactivity from the cells can include gamma counting, PET, or SPECT.

The population of cells can be exposed to the detectable substrate in the presence of a multi-drug efflux pump inhibitor with dual inhibitory action against ABCB1 and ABCG2. Exposing the population of cells to the detectable substrate can include administering the detectable substrate to a subject. The subject can be a mammal.

In another aspect, a method of treating tumor includes administering a therapeutically effective amount of a compound as described above, where *R includes a therapeutically effective radioisotope.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

Having thus described the presently disclosed subject matter in general terms, reference will now be made to the accompanying Figures, which are not necessarily drawn to scale, and wherein:

FIG. 1 shows radio-uptake assay of ¹²⁵IBz-A with K562 and L1210/cpa cells. The x-axis represents cell lines and treatments. The y-axis represents radioactivity (CPM). CPM: counts per minute, DEAB: diethylaminobenzaldehyde; and

FIG. 2 is a radiochromatogram of [¹²⁵] IBz-A.

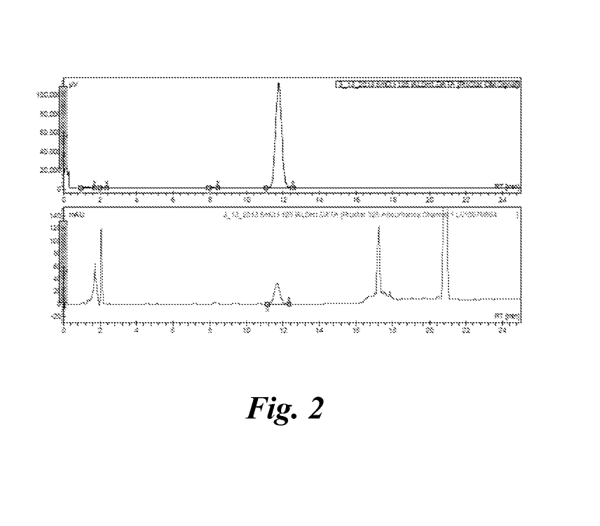

### DETAILED DESCRIPTION

The presently disclosed subject matter now will be described more fully hereinafter with reference to the accompanying Figures, in which some, but not all embodiments of the presently disclosed subject matter are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

ALDH has been studied as a marker for normal and cancer stem cells. For example, ALDH1 has been identified as a marker used to isolate cancer stem cells of various human malignancies including bladder, breast, cervical, colon, head and neck, liver, lung, pancreas, prostate, and ovary. (I. Ma and A. L. Allan, Stem Cell Rev 7 (2), 292 (2011), which is incorporated by reference in its entirety). For example, Aldh1a1 and Aldh3a1 have been implicated in the protection of stem cells from cytotoxic drugs. ALDHP^{pos} stem cells have been used as resources for regenerative (see, e.g., A. E. Balber, Stem Cells 29 (4), 570 (2011), which is incorporated by reference in its entirety). Furthermore, the presence of ALDH^{int} leukemic stem cells can be used as a predictor for relapse after therapy J. M. Gerber, B. D. Smith, B. Ngwang et al., Blood 119 (15), 3571 (2012), which is incorporated by reference in its entirety.

Detectable ALDH substrates allow those cells expressing ALDH (e.g., certain types of stem cells) in a mixed population to be distinguished from those cells that do not express ALDH. One approach is to use a compound having a detectable fluorescent moiety linked to an aldehyde group; the aldehyde group serves as a substrate for ALDH. See, for example, U.S. Pat. Nos. 5,876,956, and 6,991,897, each of which is incorporated by reference in its entirety. Another approach is to use a radiolabeled substrate. See, for example, Vaidyanathan, G., et al., “Targeting aldehyde dehydrogenase: a potential approach for cell labeling,” Nuclear Medicine and Biology 36 (2009), 919-929, which is incorporated by reference in its entirety. Both of these approaches rely on a cell-permeable aldehyde substrate being converted to a non-cell permeable carboxylic acid form. When the converted carboxylic acid form is not cell-permeable, the product, with detectable moiety, accumulates in ALDHP^{pos} cells. A related approach involves a radiolabeled compound that shows better uptake by ALDH-expressing cells than by other cells (but is not a substrate of ALDH) and accumulates in ALDHP^{pos} cells. See, e.g., Chin, B. B., et al., “Synthesis and Preliminary Evaluation of n.c.a. Iodoquine: A Novel Radiotracer with High Uptake in Cells with High ALDH1 Expression,” Current Radiopharmaceuticals 5 (2012), 47-58, which is incorporated by reference in its entirety. In some cases, the substrate also can allow cells that express ALDH to a high degree to be distinguished from cells that express it to a smaller degree.

The substrates can be used in a method for identifying intact, viable cells within a cell mixture that express an intracellular marker, for instance an enzyme such as cytosolic ALDH. The intracellular marker reacts with a cell-permeable labeled aldehyde to render the labeled substrate polar (i.e., where the aldehyde is converted to the corresponding carboxylic acid), and, hence, non-permeable to the cell membrane.

Accordingly, a method of detecting ALDHP^{pos} cells includes contacting a cell mixture with a cell-permeable, non-polar labeled aldehyde-containing substrate that is rendered polar by contact with the intracellular marker, for instance by oxidation. Once rendered polar, the labeled substrate is no longer permeable to the cell membrane and, hence, is trapped within only those cells in the cell mixture that express the intracellular marker. Cells containing the trapped polar, non-permeable labeled substrate are identified using methods and equipment of detecting radioactivity known to those of skill in the art.

The extent to which the substrate accumulates within a given cell can be related to the extent to which that cell expresses ALDH. All other things being equal, a cell expressing more ALDH will accumulate more.

The method for identifying cells containing cytosolic ALDH in intact, viable cells can provide a cell population enriched in hematopoietic stem cells, preferably a cell suspension of pluripotent hematopoietic stem cells (pluripotent HSCs), that is substantially free of lineage-committed cells. By definition “pluripotent” hematopoietic stem cells are those stem cells having the ability to repopulate all hematopoietic lineages on a long-term basis. Further discussion of isolation of pluripotent can be found, for example, in U.S. Pat. Nos. 5,876,956 and 6,991,897; and in M. Rovira, S. G. Scott, A. S. Liss et al., Proc Natl Acad Sci USA 96, 9118-9123; and Ma and A. L. Allan, Stem Cell Rev 7 (2), 292 (2011); each of which is incorporated by reference in its entirety.

Accordingly, there is a need for detectable ALDH substrates, including detectable ALDH substrates suitable for in vivo imaging of ALDHP^{pos} cells.

A compound of formula (I) can serve as a detectable substrate for ALDH:

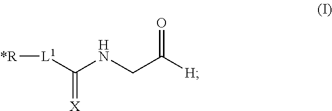

where X is 0 or S; L¹ is a linker moiety; and *R is a radiolabeled moiety. The radiolabeled moiety includes one or more radioisotopes. Specific exemplary radioisotopes include ¹⁸F, ¹²³I, ¹²⁴I, ¹²⁵I, ¹²⁶I, ¹³¹I, ⁷⁵Br, ⁷⁶Br, ⁷⁷Br, ⁸⁰Br, ^{80m}Br, ⁸²Br, ⁸³Br and ²¹¹At. Radioisotope-containing compounds can be prepared with sufficient radiolabel to be used in imaging applications. In other words, the compounds can be prepared with radioisotope concentrations greater than natural abundance, when a particular radioisotope occurs naturally.

When exposed to ALDH, the detectable substrate is converted to the corresponding carboxylic acid of formula (II):

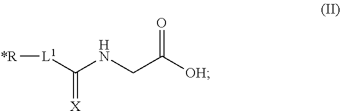

where X, L¹ and *R as defined as above. While the compound of formula (I) is desirably cell-permeable, the compound of formula (II) is desirably non-cell permeable.

In some cases, L¹ can be a bond; a C₁-C₁₀ alkylene moiety optionally substituted with from one to five substituents individually selected from H, halogen, alkyl, cycloalkyl, —OH, —O-alkyl, nitro, cyano, aryl, or heteroaryl, and optionally interrupted by one to three groups individually selected from —O—, —S—, —C(O)—, —C(S)—, —N(R^{a})—, and —C(O)N(R^{a})—; or a C₁-C₁₀ alkenylene moiety optionally substituted with from one to five substituents individually selected from H, halogen, alkyl, cycloalkyl, —OH, —O-alkyl, nitro, cyano, aryl, or heteroaryl, and optionally interrupted by one to three groups individually selected from —O—, —S—, —C(O)—, —C(S)—, —N(R^{a})—, and —C(O)N(R^{a})—; wherein each R^{a}, individually, is H, alkyl, or aryl.

In some cases, *R has the formula:

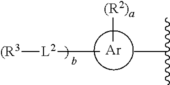

where each R², individually, can be H, halogen, —OH, nitro, cyano, alkyl, —O-alkyl, alkenyl, alkynyl, cycloalkyl, heterocyclyl, aryl, or heteroaryl, where each of alkyl, alkenyl, alkynyl, cycloalkyl, heterocyclyl, aryl, and heteroaryl can be optionally substituted by one to five substituents independently selected from H, halogen, —OH, nitro, cyano, alkyl, —O-alkyl, alkenyl, or R³ can be 0, 1, 2, 3, 4, or 5.

L² can be a bond;

a C₁-C₁₀ alkylene moiety optionally substituted with from one to five substituents individually selected from H, halogen, alkyl, cycloalkyl, —OH, —O— alkyl, nitro, cyano, aryl, or heteroaryl, and optionally interrupted by one to three groups individually selected from —O—, —S—, —C(O)—, —C(S)—, —N(R^{a})—, and —C(O)N(R^{a})—;

a C₁-C₁₀ alkenylene moiety optionally substituted with from one to five substituents individually selected from H, halogen, alkyl, cycloalkyl, —OH, —O-alkyl, nitro, cyano, aryl, or heteroaryl, and optionally interrupted by one to three groups individually selected from —O—, —S—, —C(O)—, —C(S)—, —N(R^{a})—, and —C(O)N(R^{a})—; or

a C₆-C₁₀ arylene moiety optionally substituted with from one to five substituents individually selected from H, halogen, alkyl, cycloalkyl, —OH, —O-alkyl, nitro, cyano, aryl, or heteroaryl;

where each R^{a}, individually, is H, alkyl, or aryl.

Each R³ can be a radioisotope. b can be 1, 2, 3, 4 or 5.

In some embodiments, a detectable substrate can be prepared by providing a precursor having a suitable leaving group, and substituting the leaving group with a desired radioisotope. The precursor can have a protecting group in place of the aldehyde moiety; for example, the aldehyde can be protected in the form of an acetal. After the leaving group has been replaced with the desired radioisotope, the acetal can be converted to corresponding aldehyde, affording the detectable substrate. For example, a detectable substrate can be prepared according to the following general scheme:

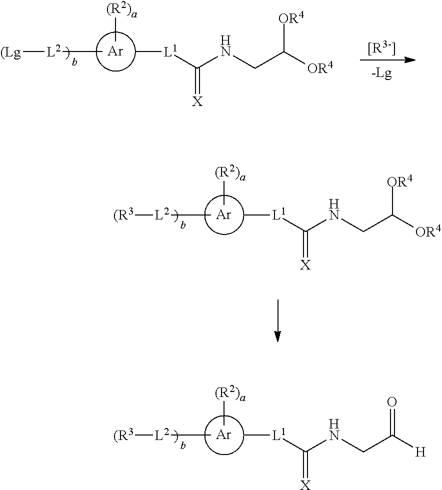

In some cases, for example, when L² is a bond, Lg is —Sn(alkyl)₃, then a suitable salt of R³⁻ can be used to install the R³ radioisotope. Some suitable R³ salts include: Na[¹²⁵I], Na [¹³¹I], Na[¹²³I], Na[¹²⁴I], K[¹⁸F], Na[⁷⁶Br], Na[⁷⁵Br], Na[²¹¹At].

In some embodiments, the detectable substrate has the formula:

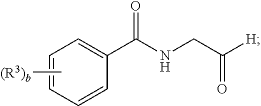

where each R³ is a radioisotope, and b is 1, 2, 3, 4, or 5.

The term “alkyl” used alone or as part of a larger moiety (i.e. “alkoxy,” “hydroxyalkyl,” “alkoxyalkyl,” and “alkoxycarbonyl”) includes both straight and branched chains containing one to ten carbon atoms (i.e. 1, 2, 3, 4, 5, 6, 7, 8, 9, or 10 carbon atoms). Examples of alkyl groups include methyl (Me), ethyl (Et), propyl (Pr) (including n-propyl (ⁿPr or n-Pr), isopropyl (ⁱPr or i-Pr), butyl (Bu) (including n-pentyl) (ⁿBu or n-Bu), isobutyl (ⁱBu or i-Bu), and tert-butyl (^{t}Bu or t-Bu)), pentyl (Pe) (including n-pentyl) and so forth. An alkyl group may be optionally substituted by 1 one 6 substituents selected from halo, hydroxyl, thiol, oxo, amino, alkylamino, dialkylamino, cyano, nitro, alkyl, alkoxy, alkenyl, alknyl, cycloalkyl, heterocyclyl, aryl, or heteroaryl.

The term “alkenyl” used along or as part of a larger moiety includes both straight and branched chains containing at least one double bond and two to ten carbon atoms (i.e., 2, 3, 4, 5, 6, 7, 8, 9, or 10 carbon atoms), as well as cyclic, non-aromatic alkenyl groups, such as cyclopropenyl, cyclobutenyl, cyclopentenyl, cyclopentadienyl, cyclohexenyl, cyclohexadienyl, and the like. As used herein, alkenyl groups also include mixed cyclic and linear alkyl groups, such as cyclopentenylmethyl, cyclopentenylethyl, cyclohexenylmethyl, and the like, so long as the total number of carbon atoms is not exceeded. When the total number of carbons allows (i.e., more than 4 carbons), an alkenyl group may have multiple double bonds, whether conjugated or non-conjugated, but do not include aromatic structures. Examples of alkenyl groups include ethenyl, propenyl, butenyl, butadienyl, isoprenyl, dimethylallyl, geranyl and so forth. An alkenyl group may be optionally substituted by 1 one 6 substituents selected from halo, hydroxyl, thiol, oxo, amino, alkylamino, dialkylamino, cyano, nitro, alkyl, alkoxy, alkenyl, alkynyl, cycloalkyl, heterocyclyl, aryl, or heteroaryl.

The term “alkynyl” used alone or as part of a larger moiety includes straight and branched chains groups containing at least one triple bond and two to ten carbon atoms (i.e., 2, 3, 4, 5, 6, 7, 8, 9, or 10 carbon atoms). When the total number of carbon atoms allows (i.e., more than 4 carbons), an alkynyl group may have multiple triple bonds, whether conjugated or non-conjugated, but do not include aromatic structures. An alkynyl group can include more than one type of multiple bond, i.e., an alkynyl group can include one or more double bonds in addition to at least one triple bond. Examples of alkenyl groups include ethynyl, propynyl, but-2-yn-yl, but-3-ynyl, and so on. An alkynyl group may be optionally substituted by 1 one 6 substituents selected from halo, hydroxyl, thiol, oxo, amino, alkylamino, dialkylamino, cyano, nitro, alkyl, alkoxy, alkenyl, alkynyl, cycloalkyl, heterocyclyl, aryl, or heteroaryl. The term “cycloalkyl” includes mono-, bi-, or tricyclic non-aromatic carbocyclic ring systems having from 3 to 14 ring carbons, and optionally one or more double bonds. The ring systems may be fused, bridged, or spiro ring systems, or a combination of these. Examples of cycloalkyl groups include saturated monocyclic groups such as cyclopropyl, cyclobutyl, cyclopentyl, cyclohexyl, cycloheptyl, cyclohexyl, cycloheptyl, cyclooctyl, and the like; unsaturated monocyclic groups such as cyclopentenyl, cyclohexenyl, cycloheptenyl, cyclopentadienyl, cyclohexadienyl, cycloheptadienyl, cyclooctatetraenyl, and the like. Examples of cycloalkyl groups also include saturated bicyclic groups such as decahydronaphthalene, bicycle[3.1.1] heptyl, norbornane, bicyclo[2.2.2]octyl, and the like; unsaturated bicyclic groups such as norbornenyl, bicyclo[2.2.2]oct-2-enyl, and the like. Examples cycloalkyl groups also include saturated tricyclic groups such as tetradecahydroanthracene, tetradecachydrophenanthrene, dodecahydro-s-indacene, and the like, and unsaturated tricyclic groups. Also included within the scope of the term “cycloalkyl” are spiro ring systems, such as spiro[4.4]nonyl, spiro[4.5]decyl, spiro[5.5]undecyl, spiro[4.6]undecyl, and the like. Also included within the scope of the term “cycloalkyl” is a group in which a non-aromatic carbocyclic ring is fused to one or more aromatic or non-aromatic rings, such as in a tetrahydronaphthyl or indanyl group, where the radical or point of attachment is on the non-aromatic carbocyclic ring. A cycloalkyl group may be optionally substituted by 1 one 6 substituents selected from halo, hydroxyl, thiol, oxo, amino, alkylamino, dialkylamino, cyano, nitro, alkyl, alkoxy, alkenyl, alkenyl, cycloalkyl, heterocyclyl, aryl, or heteroaryl.

The term “heterocycle”, “heterocyclyl”, or “heterocyclic” unless otherwise indicated includes mono-, bi-, or tricyclic non-aromatic ring systems having five to fourteen members, preferably five to ten, in which one or more ring carbons, preferably one to four, are each replaced by a heteroatom such as N, O, or S. Examples of heterocyclic groups include 3-1H-benzimidazol-2-one, (1-substituted)-2-oxo-benzimidazol-3-yl, 2-tetrahydrofuranyl, 3-tetrahydrofuranyl, 2-tetrahydropyranyl, 3-tetrahydropyranyl, 4-tetrahydropyranyl, [1,2]-dioxalanyl, [1,3]-dithiolanyl, [1,3]-dioxanyl, 2-tetrahydrothiophenyl, 3-tetrahydrothiophenyl, 2-morpholinyl, 3-morpholinyl, 4-morpholinyl, 2-thiomorpholinyl, 3-thiomorpholinyl, 4-thiomorpholinyl, 1-pyrrolidinyl, 2-pyrrolidinyl, 3-pyrrolidinyl, 1-piperazinyl, 2-piperazinyl, 1-piperidinyl, 2-piperidinyl, 3-piperidinyl, 4-piperidinyl, 4-thiazolidinyl, diazolonyl, N-substituted diazolonyl, 1-phthalimidinyl, benzoxanyl, benzopyrrolidinyl, benzopiperidinyl, benzoxolanyl, benzothiolanyl, and benzothianyl. Also included within the scope of the term “heterocyclyl” or “heterocyclic”, as it is used herein, is a group in which a non-aromatic heteroatom-containing ring is fused to one or more aromatic or non-aromatic rings, such as in an indolinyl, chromanyl, phenanthridinyl, or tetrahydroquinolinyl, where the radical or point or attachment is on the non-aromatic heteroatom-containing ring. The term “heterocycle”, “heterocyclyl”, or “heterocyclic” whether saturated or partially unsaturated, also refers to rings that are optionally substituted.

The term “aryl” used alone or as a part of a larger moiety, refers to mono-, bi-, or tricyclic aromatic hydrocarbon ring systems having five to fourteen members, such as phenyl, 1-naphthyl, 2-naphthyl, 1-anthracyl and 2-anthracyl. The term “aryl” may be used interchangeably with the term “aryl ring”. “Aryl” also includes fused polycyclic aromatic ring systems in which an aromatic ring is fused to one or more rings. Examples include 1-naphthyl, 2-naphthyl, 1-anthracyl and 2-anthracyl. Also included within the scope of the term “aryl”, as it is used herein, is a group in which an aromatic ring is fused to one or more non-aromatic rings, such as in an indanyl, phenanthridinyl or tetrahydronaphthyl, where the radical or point of attachment is on the aromatic ring.

The term “heteroaryl”, used alone or as part of a larger moiety, refers to heteroaromatic ring groups having five to fourteen members, preferably five to ten, in which one or more ring carbons, preferably one to four, are each replaced by a heteroatom such as N, O, or S. Examples of heteroaryl rings include 2-furanyl, 3-furanyl, N-imidazolyl, 2-imidazolyl, 4-imidazolyl, 5-imidazolyl, 3-isoxazolyl, 4-isoxazolyl, 5-isoxazolyl, 2-oxadiazolyl, 5-oxadiazolyl, 2-oxazolyl, 4-oxazolyl, 5-oxazolyl, 1-pyrrolyl, 2-pyrrolyl, 3-pyrrolyl, 1-pyrazolyl, 3-pyrazolyl, 4-pyrazolyl, 2-pyridyl, 3-pyridyl, 4-pyridyl, 2-pyrimidyl, 4-pyrimidyl, 5-pyrimidyl, 3-pyridazinyl, 2-thiazolyl, 4-thiazolyl, 5-thiazolyl, 5-tetrazolyl, 2-triazolyl, 5-triazolyl, 2-thienyl, 3-thienyl, carbazolyl, benzimidazolyl, benzothienyl, benzofuranyl, indolyl, quinolinyl, benzotriazolyl, benzothiazolyl, benzooxazolyl, benzimidazolyl, isoquinolinyl, indazolyl, isoindolyl, acridinyl, or benzoisoxazolyl. Also included within the scope of the term “heteroaryl”, as it is used herein, is a group in which heteroaromatic ring is fused to one or more aromatic or nonaromatic rings where the radical or point of attachment is on the heteroaromatic ring. Examples include tetrahydroquinolinyl, tetrahydroisoquinolinyl, and pyrido[3,4-d]pyrimidinyl. The term “heteroaryl” may be used interchangeably with the term “heteroaryl ring” or the term “heteroaromatic.”

Compounds of formula (I), in particular various radiolabeled compounds, may be used for diagnostic, imaging, or therapeutic purposes. For example, some compounds, e.g. those labeled with ¹²⁵I and ¹²³I, can be used for SPECT imaging, while some compounds, e.g. those labeled with ¹⁸F and ¹²⁴I, can be used for PET imaging, and some radioisotopically labeled compounds may be used therapeutically. In general, the suitability of a particular radioisotope for a particular purpose is well understood in the art. Other exemplary embodiments are compounds used as precursors for radiolabeled compounds, in which a substituent may be directly exchanged for a radioisotope in one or more steps. Unless described otherwise, the terms “converted,” “derivatized,” “exchanged,” or “reacted” are intended to encompass one or more steps. Examples of substituents that may be exchanged for radioisotopes include halogens, —NO₂, —N⁺(alkyl)₃, —Sn(alkyl)₃, —Si(alkyl)₃, —Hg(alkyl), and B(OH)₂. Other compounds are precursors which may be chemically reacted with a radioisotopically labeled reagent to produce a stable radioisotopically labeled compound. Compounds bearing substituents such as halogen, —NH₂, —NHNH₂, —Sn(alkyl)₃, and B(OH)₂, for example, may be converted into radioisotopically labeled compounds by chemical reactions known to those in the art.

Other embodiments include methods of imaging one or more cells, organs or tissues, where the method includes exposing cells to, or administering to a subject, an effective amount of a compound with an isotopic label suitable for imaging. In still another embodiment, the imaging method is suitable for imaging of cancer, tumor or neoplasm. In a further embodiment, the cancer is selected from eye or ocular cancer, rectal cancer, colon cancer, cervical cancer, prostate cancer, breast cancer and bladder cancer, oral cancer, benign and malignant tumors, stomach cancer, liver cancer, pancreatic cancer, lung cancer, corpus uteri, ovary cancer, prostate cancer, testicular cancer, renal cancer, brain cancer (e.g., gliomas), throat cancer, skin melanoma, acute lymphocytic leukemia, acute myelogenous leukemia, Ewing's Sarcoma, Kaposi's Sarcoma, basal cell carcinoma and squamous cell carcinoma, small cell lung cancer, choriocarcinoma, rhabdomyosarcoma, angiosarcoma, hemangioendothelioma, Wilms Tumor, neuroblastoma, mouth/pharynx cancer, esophageal cancer, larynx cancer, lymphoma, neurofibromatosis, tuberous sclerosis, hemangiomas, and lymphangiogenesis.

The imaging methods are suitable for imaging any physiological process or feature in which ALDH is involved. Typically, imaging methods are suitable for identification of areas of tissues or targets which express high concentrations of ALDH. In certain embodiments, the radiolabeled compound is detected by positron emission tomography (PET) or single photon emission computed tomography (SPECT).

The subject in the imaging method is a human, rat, mouse, cat, dog, horse, sheep, cow, monkey, avian, or amphibian. Typical subjects to which compounds of the invention may be administered will be mammals, particularly primates, especially humans. For veterinary applications, a wide variety of subjects will be suitable, e. g. livestock such as cattle, sheep, goats, cows, swine and the like; poultry such as chickens, ducks, geese, turkeys, and the like; and domesticated animals particularly pets such as dogs and cats. For diagnostic or research applications, a wide variety of mammals will be suitable subjects including rodents (e.g. mice, rats, hamsters), rabbits, primates, and swine such as inbred pigs and the like. Additionally, for in vitro applications, such as in vitro diagnostic and research applications, body fluids and cell samples of the above subjects will be suitable for use such as mammalian, particularly primate such as human, blood, urine or tissue samples, or blood urine or tissue samples of the animals mentioned for veterinary applications. The body fluids and cell samples of the above subjects can be in vivo or in vitro.

In certain of the presently disclosed methods, the compounds of the invention are excreted from tissues of the body quickly to prevent prolonged exposure to the radiation of the radiolabeled compound administered to the patient. Typically compounds of the invention are eliminated from the body in less than about 24 hours. More typically, compounds of the invention are eliminated from the body in less than about 16 hours, 12 hours, 8 hours, 6 hours, 4 hours, 2 hours, 90 minutes, or 60 minutes. Exemplary compounds are eliminated in between about 60 minutes and about 120 minutes.

Other embodiments provide methods of treating tumors comprising administering to a subject a therapeutically effective amount of a compound of formula (I) comprising a therapeutically effective radioisotope. In certain embodiments, the tumor cells may express ALDH. In other embodiments, a tumor may be treated by targeting adjacent or nearby cells which express ALDH. Examples of therapeutically effective radioisotopes include ¹³¹I and ²¹¹At.

Stem cells generally express one or more active multi-drug efflux pumps, such as ABCB1 and/or ABCG2. The detectable ALDH substrate may also be a substrate for those pumps. Thus, even though ALDH substrates are converted into polar form (e.g., carboxylic acid form), the pumps can remove the converted ALDH from the cell, contrary to the desired accumulation of the converted ALDH substrate within ALDHP^{pos} cells. It can therefore be desirable, when assaying cells for ALDH activity, to include an inhibitor of one or more multi-drug efflux pumps. For example, the commercial Aldefluor® assay buffer contains verapamil, a pump inhibitor (see also U.S. Pat. No. 6,991,897, which is incorporated by reference in its entirety). Verapamil is an inhibitor of ABCB1, but does not inhibit ABCG2. Even in the presence of verapamil, cells that do accumulate the converted substrate can exhibit gradual decrease of fluorescent intensity over time (e.g., on the order of 1 hour). Inhibiting both pumps can enhance the accumulation of the converted inhibitor in ALDHP^{pos} cells, so that identification of ALDHP^{pos} cells is more effective than when no inhibitor, or an inhibitor of only one pump, is present.

Therefore it can be advantageous to carry out assays for ALDHP^{pos} cells in the presence of an inhibitor of ABCB1, and inhibitor of ABCG2, or more preferably in the presence of both an inhibitor of ABCB1 and an inhibitor of ABCG2, or more preferably in the presence of a dual-activity inhibitor of ABCB1 and ABCG2, i.e., a single compound that inhibits both ABCB1 and ABCG2. Inhibitors of ABCB1, including verapamil, are known. Some inhibitors of ABCG2 are described in, for example, Zhang, Y., et al., Cancer Res. 2009; 69 (14), 5867-5875, which is incorporated by reference in its entirety. Dual action inhibitors, i.e., that inhibit both ABCB1 and ABCG2, include Galfenine, doxazosin mesylate, clebopride maleate, and flavoxate hydrochloride. Inhibitors of ABCG2 (but not ABCB1) include: fumitremorgin C (FTC), Ko143, Gefitinib, Harmine, Prazosin, Dipyridamole, Curcumin, Nelfinavir mesylate, Niguldipine, Riboflavin, Reserpine, Hesperetin, Tracazolate, Verteporfin, Quinacrine, Metyrapone, Rotenone, Acepromazine, Flutamide, Podophyllum resin, Piperacetazine, Acetophenazine maleate, and Raloxifine hydrochloride.

Other embodiments provide kits comprising a compound of formula (I). In certain embodiments, the kit provides packaged pharmaceutical compositions comprising a pharmaceutically acceptable carrier and a compound of formula (I). In certain embodiments the packaged pharmaceutical composition will include the reaction precursors necessary to generate the compound of formula (I) upon combination with a radiolabeled precursor. Other packaged pharmaceutical compositions further include indicia comprising at least one of: instructions for preparing compounds according to the invention from supplied precursors, instructions for using the composition to image cells or tissues expressing ALDH.

In certain embodiments, a kit according to the invention contains from about 1 to about 30 mCi of the radionuclide-labeled compound described above, in combination with a pharmaceutically acceptable carrier. The compound and carrier may be provided in solution or in lyophilized form. When the compound and carrier of the kit are in lyophilized form, the kit may optionally contain a sterile and physiologically acceptable reconstitution medium such as water, saline, buffered saline, and the like. The kit may provide a compound in solution or in lyophilized form, and these components of the kit may optionally contain stabilizers such as NaCl, silicate, phosphate buffers, ascorbic acid, gentisic acid, and the like. Additional stabilization of kit components may be provided in this embodiment, for example, by providing the reducing agent in an oxidation-resistant form. Determination and optimization of such stabilizers and stabilization methods are well within the level of skill in the art.

In certain embodiments, a kit provides a non-radiolabeled precursor to be combined with a radiolabeled reagent on-site. Examples of radioactive reagents include Na[¹²⁵I], Na[¹³¹I], Na[¹²³I], Na[¹²⁴I], K[¹⁸F], Na[⁷⁶Br], Na[⁷⁵Br], Na[²¹¹At].

Imaging agents may be used to generate images by virtue of differences in the spatial distribution of the imaging agents which accumulate at a site when contacted with converted to polar form by ALDH. The spatial distribution may be measured using any means suitable for the particular label, for example, a gamma camera, a PET apparatus, a SPECT apparatus, and the like. The extent of accumulation of the imaging agent may be quantified using known methods for quantifying radioactive emissions.

In general, a detectably effective amount of the imaging agent of the invention is administered to a subject. In accordance with the invention, “a detectably effective amount” of the imaging agent of the invention is defined as an amount sufficient to yield an acceptable image using equipment which is available for clinical use. A detectably effective amount of the imaging agent of the invention may be administered in more than one injection. The detectably effective amount of the imaging agent of the invention can vary according to factors such as the degree of susceptibility of the individual, the age, sex, and weight of the individual, idiosyncratic responses of the individual, and the dosimetry. Detectably effective amounts of the imaging agent of the invention can also vary according to instrument and film-related factors. Optimization of such factors is well within the level of skill in the art. The amount of imaging agent used for diagnostic purposes and the duration of the imaging study will depend upon the radionuclide used to label the agent, the body mass of the patient, the nature and severity of the condition being treated, the nature of therapeutic treatments which the patient has undergone, and on the idiosyncratic responses of the patient. Ultimately, the attending physician will decide the amount of the imaging agent to administer to each individual patient and the duration of the imaging study.

A “pharmaceutically acceptable carrier” refers to a biocompatible solution, having due regard to sterility, p[Eta], isotonicity, stability, and the like and can include any and all solvents, diluents (including sterile saline, Sodium Chloride Injection, Ringer's Injection, Dextrose Injection, Dextrose and Sodium Chloride Injection, Lactated Ringer's Injection and other aqueous buffer solutions), dispersion media, coatings, antibacterial, and antifungal agents, isotonic agents, and the like. The pharmaceutically acceptable carrier may also contain stabilizers, preservatives, antioxidants, or other additives, which are well known to one of skill in the art, or other vehicle as known in the art.

As used herein, “pharmaceutically acceptable salts” refer to derivatives of the disclosed compounds wherein the parent compound is modified by making non-toxic acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, malefic, hydroxymaleic, phenylacetic, glutamic, benzoic, salicylic, mesylic, sulfanilic, 2-acetoxybenzoic, fumaric, toluenesulfonic, methanesulfonic, ethane disulfonix, oxalic, isethionic, HOOC—(CH₂)ₙ— COOH where n is 0, 1, 2, 3, or 4, and the like. The pharmaceutically acceptable salts of the present invention can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are used, where practicable. Lists of additional suitable salts may be found, e.g., in Remington's Pharmaceutical Sciences, 17th ed., Mack Publishing Company, Easton, Pa., p. 1418 (1985).

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, parameters, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

### Methods and Materials

Cell Lines.

The K562 human chronic myelogenous leukemia cell line was purchased from American Type Culture Collection (CLL-243™) and maintained in suspension in IMDM media supplemented with 10% FBS. The murine leukemia cell line L1210 (ALDH^{low}) and L1210/cpa (ALDH^{hi}) were provided by Dr. Richard J. Jones (Johns Hopkins University) and maintained in suspension in RPMI 1640 supplemented with 10% FBS. Human glioma cell line, U87-tri was provided by Dr. John Laterra (Kennedy Krieger Institute) and maintained in DMEM media supplemented with 10% FBS. All cells were grown at 37° C. in a humidified incubator with 5% CO₂.

Reagents and Analyses.

Chemicals and solvents obtained from commercial sources were analytical grade or better and used without further purification. Sodium Iodide-125 (Na¹²⁵I) was obtained as a 0.1 N solution of NaOH (high concentration) from MP Biomedical (Solon, Ohio). Analytical thin-layer chromatography (TLC) was performed using Aldrich aluminum backed 0.2 mm silica gel plates and visualized by UV light (254 nm) and I₂. Flash column chromatography was performed on silica gel (60 Å, MP Biomedicals). Radio-HPLC purification was performed using a Waters (Milford, Mass.) system equipped with two Waters 510 pumps, a Waters 490E variable wavelength UV/Vis detector set at 254 nm, a BioScan FlowCount radioactivity detector, a Waters radial-PAK C18 reverse phase analytical column (8×100 mm) with H₂O/CH₃CN/TFA solvent systems, and Win Flow (LabLogic) chromatography software. 1H NMR was recorded on a Bruker (Billerica, Mass.) Ultrashield™ 400 MHz spectrometer. ESI mass spectra were obtained with a Bruker Daltonics Esquire 300 plus spectrometer. Radioactivity was measured in a Capintec CRC-12 dose calibrator.

### Example 2

### Synthesis of ALDH Substrates

IBz Aldehyde Diethyl Acetal (IBz-A-DA).

To 50 mg (0.15 mmol) of N-succinimidyl 4-iodobenzoate in 2 mL of tetrahydrofuran (THF) was added 38 mg of aminoaldehyde diethyl acetal (0.3 mmol) and 50 μL of NEt₃ and stirred at room temperature for 1 hr. IBz-A-DA was purified by flash column chromatography using 2:1 hexane/ethyl acetate to give IBz-A-DA 1 (R=0.3, 47 mg, 90% yield). ¹H NMR (CDCl₃) 1.21 (t, 6H, J=6.8 hz), 3.55 (m, 4H), 3.72 (m, 2H), 4.59 (dd, 1H, J=6 hz, 4.8 hz), 6.33 (br, 1H), 7.47 (d, 2H, J=7.6 Hz), 7.76 (d, 2H, J=7.6 Hz).

The synthesis of IBz-A-DA and IBz-A is provided in Scheme 1.

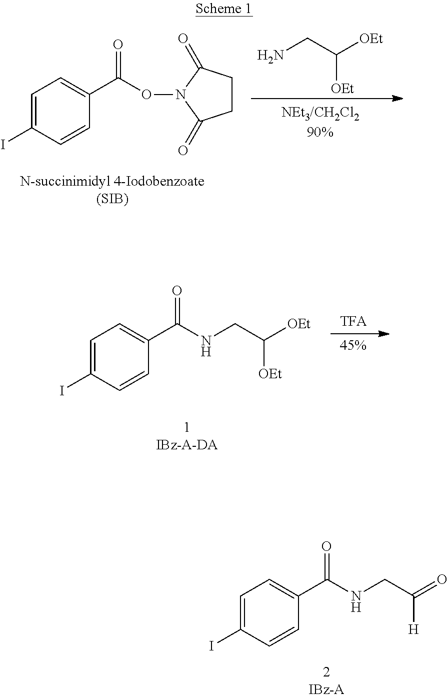 [SMILES: O=C(ON1C(=O)CCC1=O)c1ccc(I)cc1 ; CCOC(CN)OCC ; CCOC(CNC(=O)c1ccc(I)cc1)OCC ; O=CCNC(=O)c1ccc(I)cc1]

IBz aldehyde (IBz-A).

To 15 mg of IBz-A-DA was added 0.5 mL of CH₂Cl₂ was added 0.5 mL of TFA. After 0.5 hour reaction, TLC showed that all starting materials were disappeared. Reaction solvent was removed in vacuo and IBz-A was purified by flash column chromatography using 1:1 hexane/ethyl acetate to give IBz-A 2 (R_{f}=0.35, 5.3 mg, 45% yield). ¹H NMR (CDCl³) 4.45 (s, 2H), 6.86 (br, 1H), 7.57 (d, 2H, J=8 HZ), 7.84 (d, 2H, J=8 Hz), 9.81 (s, 1H).

SnBz aldehyde diethyl acetal (SnBz-A-DA).

To 32 mg of N-succinimidyl 4-(tri-n-butylstannyl)benzoate (0.062 mmol) in 1 mL of THF was added 17 mg of aminoaldehyde diethyl acetal (0.126 mmol) and 50 μL of Net₃ and stirred at room temperature for 1 hr. Then SnBz-A-DA was purified by flash column chromatography using 3:1 hexane/ethyl acetate to give SnBz-A-DA 3 (R=0.25, 30 mg, 66% yield).

### Example 3

### Radiosynthesis of ¹²⁵I-IBz-A-DA

To a 1 mL v-vial was added 50 μL of SnBz-A-DA in MeOH (1 mL/min) and 10 μL of NCS in water (1 mg/mL) and 10 μL of water, 9.8 mCi of I¹²⁵-NaI was then added. The reaction mixture was incubated at room temperature for 25 minutes, and injected into HPLC for purification. HPLC condition is 70/30 water/acetonitrile with flow rate at 1 mL/min on a waters radial Pak C18 analytical column. The fraction at retention time of 22 minutes is collected to give ¹²⁵I-IBz-A-DA (8.3 mCi, 84% RCY, Specific activity: 2000 mCi/μmol). Radioiodination of tin precursor SnBz-A-DA yielded the acetal intermediate [¹²⁵] IBz-A-DA in about 85% radiochemical yield.

The synthesis of ¹²⁵IBz-A-DA and ¹²⁵IBz-A is provided in Scheme 2. Alternative conditions for the synthesis of IBz-A-DA, and IBz-A, ¹²⁵IBz-A-DA, and ¹²⁵IBz-A are provided in Scheme 3.

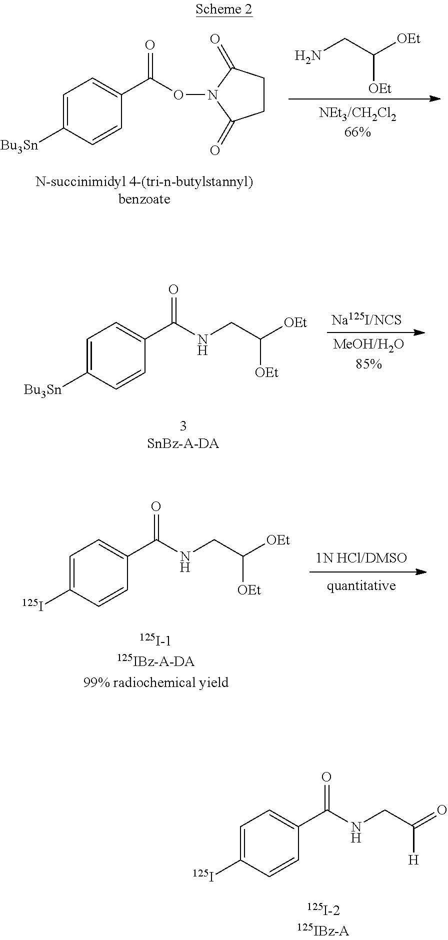

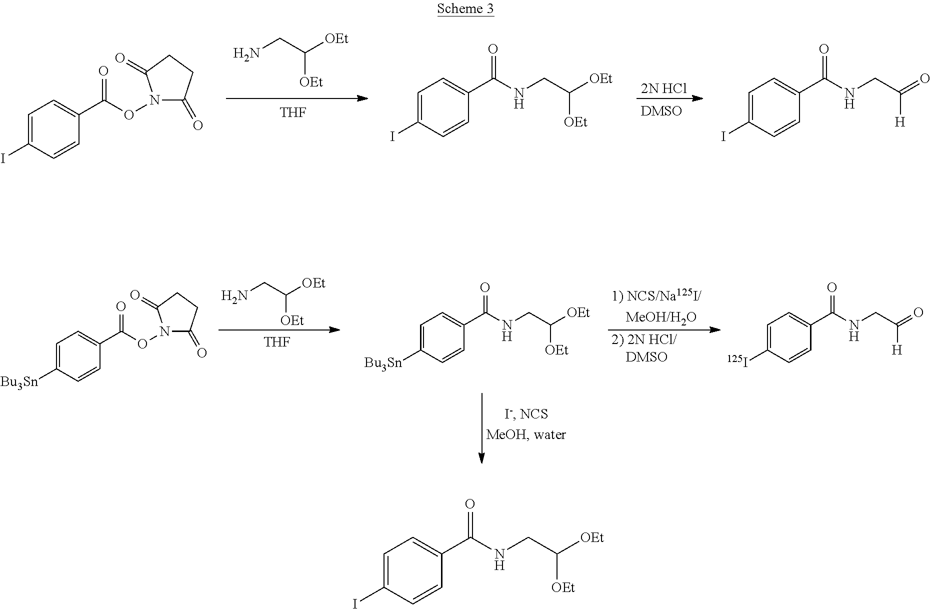

### Example 4

### Preparation of Aldehyde Forms of the Presently Disclosed Agents

¹²⁵IBz-A-DA was dissolved in 100% DMSO at 10 mCi/50 μL concentration and stored at −20° C. as stock solutions. 25 μL of a stock was deprotected by mixing with the same volume of 2N HCl for 30 mins at room temperature and the resulting aldehyde (IBz-A) were neutralized by adding 350 μL of the assay buffer (PBS supplemented with 1% FBS and 50 μM Verapamil, Sigma V4629) and immediately used.

### Example 5

### In Vitro Radio Uptake Assay for ALDH Activity

One million cells were resuspended in the assay buffer and 1 μCi of IBz-A (with or without 1000 fold cold competitor) was added to the cell. A 0.5-mL aliquot of cells was immediately taken and added to a tube containing 5 μL of DEAB (Stem Cell Technologies, 01705). Cells were incubated in 37° C. water bath for 30 mins and washed with 4 mL of the cold assay buffer. Cells were resuspended in the cold assay buffer (5×10⁵/2004) and stored in the ice until analyzed. Cells were washed twice with cold assay buffer and the radioactivity was measured by LKB Wallac gamma counter (1282 compugamma CS).

### Example 5

### Results and Discussion

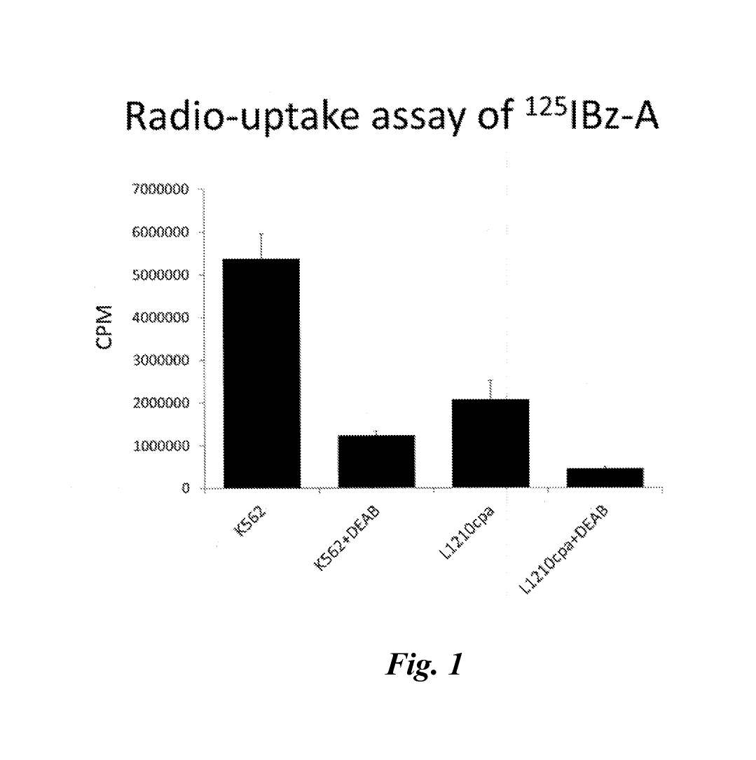

¹²⁵IBz-A was tested for specific uptake by ALDH-expressing cell lines (K562 and L1210/cpa). When compared with cells treated with DEAB (ALDH inhibitor), both cells showed more than 5-fold increase of uptake (FIG. 1). This increase in uptake indicates ¹²⁵IBz-A is a specific substrate of ALDH and can be used to label live cells that express ALDH.

Although the foregoing subject matter has been described in some detail by way of illustration and example for purposes of clarity of understanding, it will be understood by those skilled in the art that certain changes and modifications can be practiced within the scope of the appended claims.

1-6. (canceled)

7. A detectable substrate for ALDH comprising a compound having the formula: 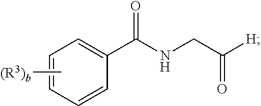
wherein each R³, independently, is a radioisotope selected from the group consisting of ¹²³I, ¹²⁴I, ¹²⁵I, ¹²⁶I, ¹³¹I, ⁷⁵Br, ⁷⁶Br, ⁷⁷Br, ⁸⁰Br, ^{80m}Br, ⁸²Br, ⁸³Br, and ²¹¹At, and b is 1, 2, 3, 4, or 5.

8. The detectable substrate of claim 7, wherein R³ is selected from the group consisting of ¹²³I, ¹²⁴I, ¹²⁵I, ¹³¹I, ²¹¹At, and b is 1.

9. The detectable substrate of claim 8, having a formula selected from the group consisting of:

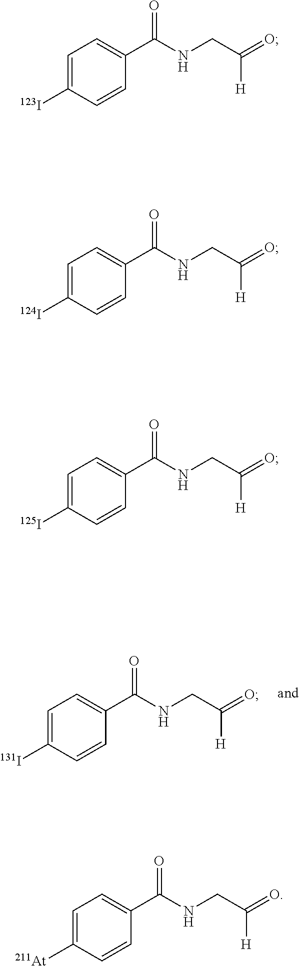

10. A method for distinguishing ALDH-expressing cells in a population of cells, comprising:
exposing the population of cells to detectable substrate for ALDH of claim 8;
measuring radioactivity from the cells; and
identifying cells exhibiting increased radioactivity from the detectable substrate.

11. The method of claim 10, further comprising, prior to measuring radioactivity from the cells:
converting the detectable substrate to the corresponding carboxylic acid within cells expressing ALDH; and
retaining and accumulating the corresponding carboxylic acid within cells expressing ALDH.

12. The method of claim 10, wherein measuring radioactivity from the cells includes gamma counting, PET, or SPECT.

13. The method of claim 10, wherein the population of cells is exposed to the detectable substrate in the presence of a multi-drug efflux pump inhibitor with dual inhibitory action against ABCB1 and ABCG2.

14. The method of claim 10, wherein exposing the population of cells to the detectable substrate includes administering the detectable substrate to a subject.

15. The method of claim 14, wherein the subject is a mammal.

16. A method for treating a tumor, comprising administering a therapeutically effective amount of a compound according to claim 8, wherein R³ includes a therapeutically effective radioisotope selected from the group consisting of ¹²³I, ¹²⁴I, ¹²⁵I, ¹²⁶I, ¹³¹I, ⁷⁵Br, ⁷⁶Br, ⁷⁷Br, ⁸⁰Br, ^{80m}Br, ⁸²Br, ⁸³Br, and ²¹¹At.
